(19) United States
(12) Patent Application Publication
(10) Pub. No.: US 2009/0111988 A1
(43) Pub. Date: Apr. 30, 2009

(54) NOVEL PROCESS FOR PRODUCTION OF 5--6-CHLORO-1,3-DIHYDRO-2H-INDOL-2-ONE (ZIPRASIDONE)

(76) Inventors: Jozsef Neu, Budapest (HU); Jozsef Torley, Budapest (HU); Sandor Garadnay, Esztergom (HU)

Correspondence Address: HAHN & VOIGHT PLLC, 1012 14TH STREET, NW, SUITE 620, WASHINGTON, DC, 20005, US

(21) Appl. No.: 12/298,590

(22) PCT Filed: May 2, 2007
(86) PCT No.: PCT/HU07/00038

§ 371 Date: Nov. 24, 2008

(30) Foreign Application Priority Data
May 2, 2006 (HU) P0600347

(51) Int. Cl. C07D 417/14; C07D 209/34

(52) U.S. Cl. 544/368; 548/486

(57) ABSTRACT

The present invention provides a novel, industrially easily realisable and economically preferable process for production of pure 5-{2-[4-(1,2-benzisothiazol-3-yl)-1-piperazinyl]-ethyl}-6-chloro-1,3-dihydro-2H-indol-2-one i.e., ziprasidone hydrochloride shown in the reaction scheme (II), (III), (IV), (V) and (VI). According to the invention the intermediate compound 5-(2-bromoethyl)-6-chloro-1,3-dihydro-2H-indol-2-one of Formula (III) is produced from 5-(2-bromoacethyl)-6-chloro-1,3-dihydro-2H-indole-2-one of Formula (IV). The highly pure ziprasidone base of Formula (II) is obtained in the reaction of 3-piperazinyl-1,2-benzisothiazol of Formula (VI) with 5-(2-bromoethyl)-6-chloro-1,3-dihydro-2H-indol-2-one of Formula (III) in an organic solvent or organic solvent mixture.

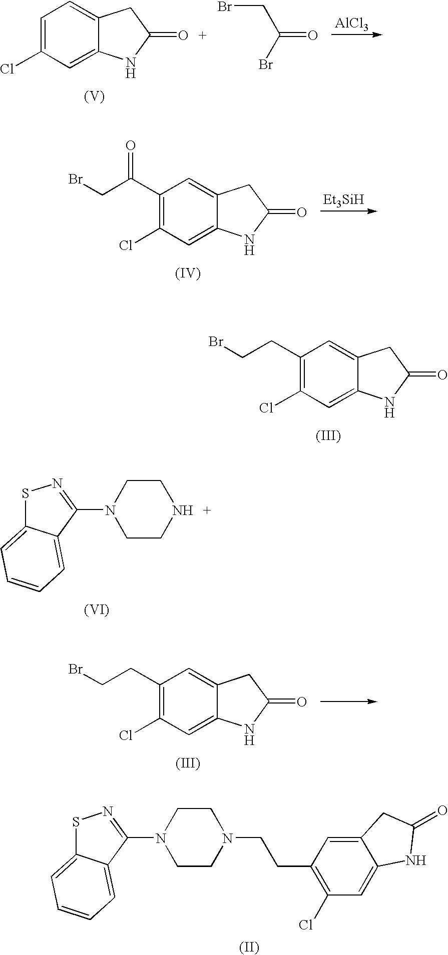

### FIELD OF THE INVENTION

The field of the invention relates to a new process for the preparation of pure ziprasidone, i.e. 5-{2-[4-(1,2-benzisothiazol-3-yl)-1-piperazinyl]-ethyl}-6-chloro-1,3 -dihydro-2H-indol-2-one. The invention also relates to an intermediate, i.e. 5-(2-bromoethyl)-6-chloro-1,3-dihydro-2H-indol-2-one, and a process for its production.

### BACKGROUND OF THE INVENTION

Ziprasidone hydrochloride, 5-{2-[4-(1,2-benzisothiazol-3-yl)-1-piperazinyl]-ethyl}-6-chloro-1,3-dihydro-2H-indol-2-one of Formula I is disclosed in U.S. Pat. No. 4,831,031 (European equivalent: EP 0 281 309) and is known as the active ingredient of neuroleptic drugs.

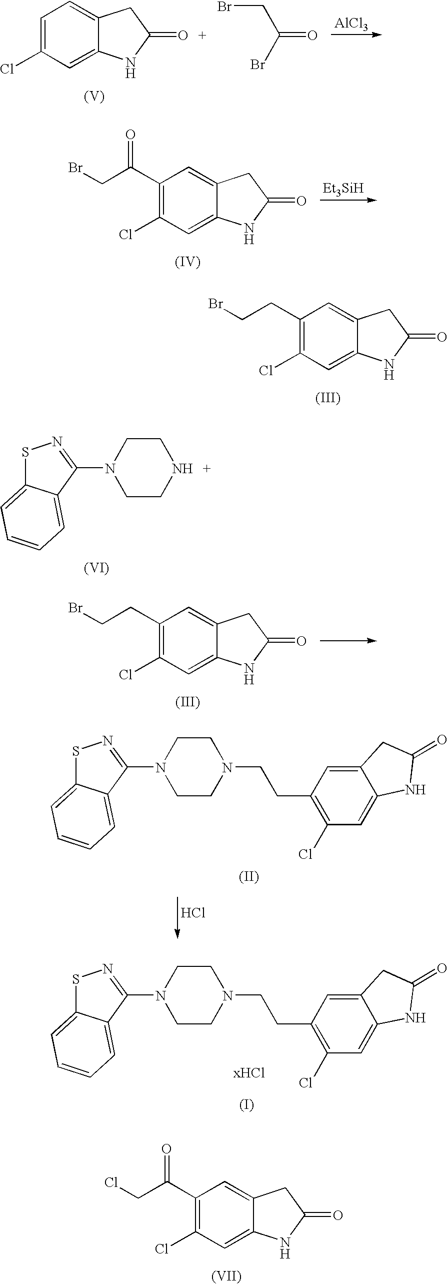

According to this patent 5-{2-[4-(1,2-benzisothiazol-3-yl)-1-piperazinyl]-ethyl}-6-chloro-1,3-dihydro-2H-indol-2-one hydrochloride of Formula I is obtained if 5-(2-chloroethyl)-6-chloro-1,3-dihydro-2H-indol-2-one of Formula VII is reacted with 3-piperazinyl-1,2-benzisothiazol hydrochloride of Formula VI in the presence of sodium-carbonate and sodium-iodide in methyl-isobutyl-ketone boiling the mixture for 40 hours. Then the reaction mixture is filtered, evaporated, and the residue is clarified with chromatography. The evaporated residue of chromatography is dissolved in dichloromethane, and after acidification by hydrochloric acidic diethyl ether, the precipitated crystals are filtered out, washed with ether, acetone.

The obtained product is declared as 5-{2-[4-(1,2-benzisothiazol-3-yl)-1-piperazinyl]-ethyl}-6-chloro-1,3-dihydro-2H-indol-2-one hydrochloride hemi hydrate (ziprasidone hydrochloride hemi hydrate).

This method is unusable for industrial production, however according to the procedure of European Patent No. EP 584 903 5-{2-[4-(1,2-benzisothiazol-3-yl)-1-piperazinyl]-ethyl}-6-chloro-1,3-dihydro-2H-indol-2-one hydrochloride of Formula I can be produced at a high yield even in an industrial scale (80%). In this procedure also the same components: 5-(2-chloroethyl)-6-chloro-1,3-dihydro-2H-indol-2-one of Formula VII and the hydrochloride salt of 3-piperazinyl-1,2-benzisothiazol of Formula VI are reacted with each other in the presence of sodium-carbonate, but in this case the solvent is simply water. Here the isolation is followed by a complicated clearing step.

European Patent No. EP 586 191 reveals a method according with 5-{2-[4-(1,2-benzisothiazol-3-yl)-1 -piperazinyl]-ethyl}-6-chloro-1,3-dihydro-2H-indol-2-one hydrochloride monohydrate (ziprasidone hydrochloride monohydrate) is obtained in a reaction of the clean ziprasidone base with diluted aqueous hydrochloric acid solution.

PCT Publication No. WO 2003/99198 has not brought significant changes about in the production procedure, however PCT Publication No. WO 2004/050655 revealed a procedure where the known compounds of Formula VI and Formula VII are reacted in the presence of sodium-iodide, sodium-carbonate and tetrabuthyl-phosphonium bromide in the solvent. The reaction mixture is boiled until end of the reaction. In our reproduction even after 72 hours the product was still only in traces in the reaction mixture. According to the procedure from the ziprasidone base amorphous 5-{2-[4-(1,2-benzisothiazol-3-yl)-1-piperazinyl]-ethyl}-6-chloro-1,3-dihydro-2H-indol-2-one hydrochloride (ziprasidone hydrochloride) is prepared in a complicated way.

According to PCT Publication No. WO 2005/040160 ,“ionic additive” (NaCl, NaSO4) is added into the known distilled water—sodium-carbonate—sodium-iodide reaction mixture, the compounds of Formula VI and Formula VII are reacted in this mixture in the known circumstances. Our investigations could not detect significant changes, the compound of Formula I is formed in the expected quantity, and according to the description in 85-95% purity.

From our experiments, it generally could be determined that none of the procedures using purely organic solvents for the reaction of Formula VI and Formula VII is sufficiently reproducible. Furthermore, during our experiments, although the reaction was accomplished both in aqueous-alcoholic and aqueous-glycerinic medium, the products in both cases were too contaminated, that prevents their use for pharmaceutical purposes, or a complicated and loss-making purification step must be inserted.

5-(2-bromoethyl)-6-chloro-1,3-dihydro-2H-indol-2-one of the Formula III is first mentioned in the PCT Publication No. WO 2005/085240 A2 without any physical parameters. This description does not inform about the source or preparation method of this compound, only its use instead of the compound of Formula VII is demonstrated. The compound 3-piperazinyl-1,2-benzisothiazol hydrochloride of Formula VI was reacted with 5-(2-bromoethyl)-6-chloro-1,3-dihydro-2H-indol-2-one of Formula III at similar conditions as in case of its chloroethyl analogue of Formula VII in the proved ionised water solution using sodium-carbonate sodium-iodide salts. They reported a good yield (70%), and sufficient purity. In our reproduction experiments much weaker results were achieved.

Surprisingly, it was found that while in case of 5-(2-chloro-ethyl)-6-chloro-1,3-dihydro-2H-indol-5-one, ziprasidone of Formula VII with appropriate quality and quantity can only be produced in an aqueous medium, and in contrary, in case of 5-(2-bromoethyl)-6-chloro-1,3-dihydro-2H-indol-2-one of Formula III the coupling reaction just in organic medium can sufficiently be accomplished. A really high purity ziprasidone with a sufficient yield can be prepared, if 5-(2-bromoethyl)-6-chloro-1,3-dihydro-2H-indol-2-on of Formula III is reacted in organic solvent with 3-piperazinyl-1,2-benzisothiazol.

### SUMMARY OF THE INVENTION

The present invention provides a novel, industrially easily realisable and economically preferable process for production of pure 5-{2-[4-(1,2-benzisothiazol-3-yl)-1-piperazinyl]-ethyl}-6-chloro-1,3-dihydro-2H-indol-2-one i.e., ziprasidone hydrochloride shown in the following reaction scheme.

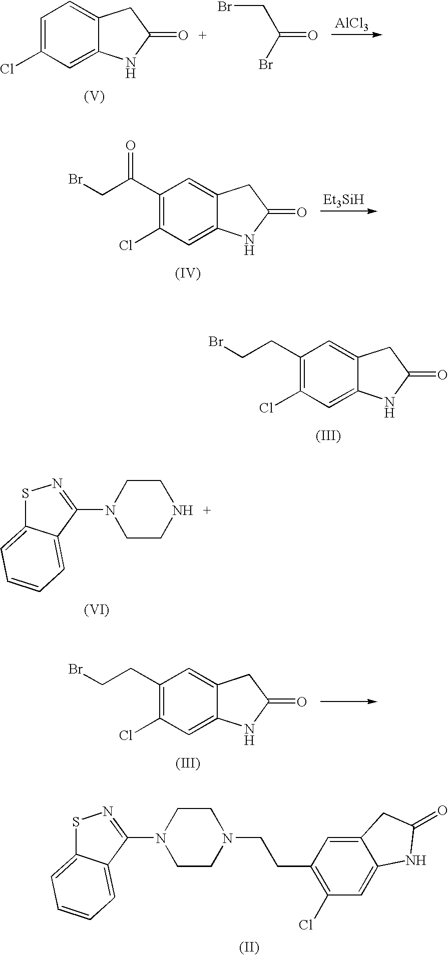

According to the invention the intermediate compound 5-(2-bromoethyl)-6-chloro-1,3-dihydro-2H-indol-2-one of Formula III is produced from 5-(2-bromoacethyl)-6-chloro-1,3-dihydro-2H-indole-2-one of Formula IV. The highly pure ziprasidone base of Formula II is obtained in the reaction of 3-piperazinyl-1,2-benzisothiazol of Formula VI with 5-(2-bromoethyl)-6-chloro-1,3-dihydro-2H-indol-2-one of Formula III in an organic solvent or organic solvent mixture.

### DETAILED DESCRIPTION OF THE INVENTION

During our experimental work we found that the 5-(2-bromoethyl)-6-chloro-1,3-dihydro-2H-indol-2-one of Formula III can really be prepared. This material was crystallised from tetrahydrofuran, and could be stored as stabile compound.

The obtained compound was characterised by the following NMR data:

1H NMR: 3.17 t (2H) [H2-12]; 3.46 s (2H) [H2-3]; 3.64 t (2H) [H2-14]; 6.83 s (1H) [H-9]; 7.24 s (1H) [H-6]; 10.46 s (1H) [NH].

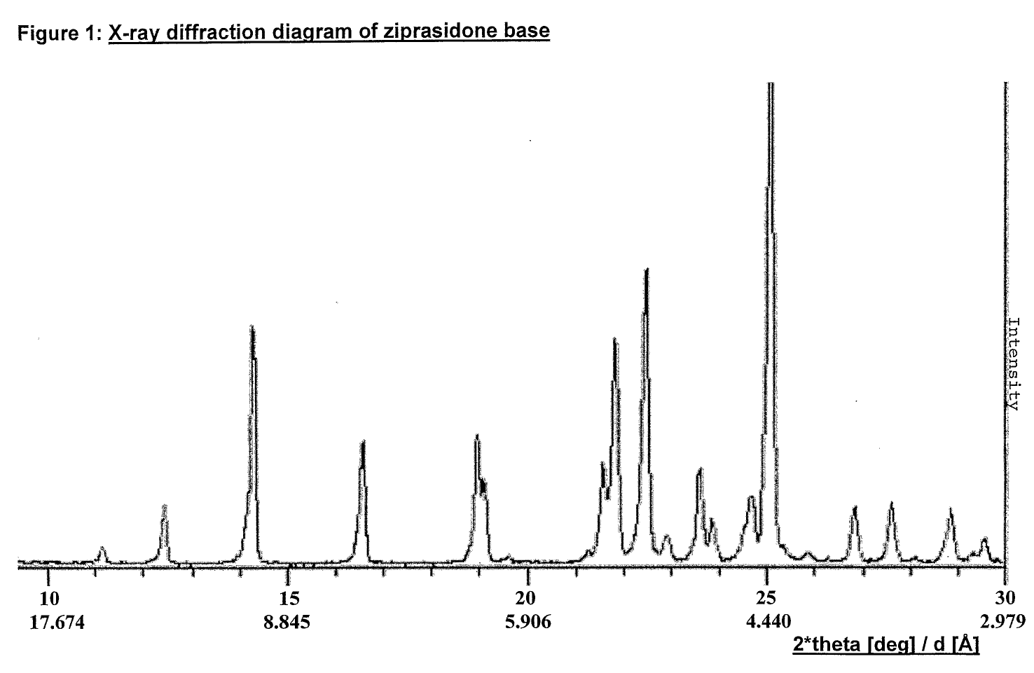

The compound was also characterised by the following X-ray diffraction 2*theta data (see the diagram in FIG. 1):

14.28 16.55 18.95 21.81 22.47 25.06.

This compound of Formula III is prepared similarly as that of 5-(2-chloro-ethyl)-6-chloro-1,3-dihydro-2H-indol-5-on of Formula VII: 6-chloro-1,3-dihydro-2H-indol-5-on of Formula V in a Friedel-Crafts type reaction is reacted with bromacethyl-bromide, and the formed 5-(2-bromo-acetyl)-6-chloro-1,3-dihydro-2H-indol-5-on of Formula (IV) is reduced by trimethyl silane. This reduction with trimethyl silane is accomplished with a high yield in the presence of a strong Bronsted-Lowry acid, e.g. trifluoroacetic acid, methane sulphonic acid, sulphuric acid, etc. or in the presence of a Lewis acid, e.g. boron trifluoride etherate, aluminium-trichloride, etc. Consequently the compound of Formula III can be prepared directly in an “in situ” reduction from 5-(2-bromo-acetyl)-6-chlor-1,3-dihydro-2H-indol-5-on of Formula IV formed in the Friedel-Crafts reaction. The reduction can be accomplished following an isolation step of Formula IV, as well.

During our experiments it was also discovered that the compound 5-{2-[4-(1,2-benzisothiazol-3 -yl)-1 -piperazinyl]-ethyl}-6-chloro-1,3 -dihydro-2H-indol-2-one (ziprasidone) can be produced in a high quality and yield if the starting material is the above-mentioned 5-(2-bromoethyl)-6-chloro-1,3-dihydro-2H-indol-2-one of Formula IV, and if advantageously organic solvents are used, that can comprise alcohohols, ketones, aliphatic and aromatic carbohydrates aliphatic nitriles and other accepted solvents in the pharmaceutical industry. Especially good results were achieved in aprotic solvents.

The compound 5-{2-[4-(1 ,2-benzisothiazol-3-yl)-1 -piperazinyl]-ethyl}-6-chloro-1,3-dihydro-2H-indol-2-one of Formula I can be produced at an especially high yield if one mol 5-(2-bromoethyl)-6-chloro-1,3-dihydro-2H-indol-2-one of Formula IV is reacted with two mol 3-piperazinyl-1,2-benzisothiazol base of Formula VI at the temperature of reflux in acetonitrile. For 2-3 hours the reaction is accomplished and the crude base can be obtained at more than 99 % purity, with a yield of 85 %.

The ziprasidone base obtained this manner is characterised by the X-ray diffraction diagram of FIG. 1.

FIG. 1. X-ray diffraction diagram of ziprasidone base

### EXAMPLES

The present invention is illustrated by the following examples without limiting the scope.

### Example 1

### Preparation of 5-(2-Bromoethyl)-6-Chloro-1,3-Dihydro-2H-Indol-2-One (III)

40g (0.3 mol) anhydrous AlCl₃ was suspended in 80 ml dichloro methane, it was cooled to a temperature between 0-10° C. and after 30 min stirring 9.6 ml (0.11 mol) bromoacethyl-bromide was added dropwise, then 16.7 g 6-chloro-1,3-dihydro-2H-indol-5-on of Formula V was added, and the reaction mixture was stirred at a room temperature for 24 hours. The accomplishment of the reaction was checked by thin-layer chromatography. 35.1 ml (0.22 mol) triethyl-silane was added dropwise to the reaction mixture and heated to the boiling point. After 30 min the mixture was poured onto ice, the precipitated material was filtered out, than it was washed three times with 40 ml water, then with 20 ml methanol, and it was dried.

19.7 g 5-(2-bromoethyl)-6-chloro-1,3-dihydro-2H-indol-2-one of Formula IV was obtained.

The material was characterised by the following NMR data:

1H NMR: 3.17 t (2H) [H2-12]; 3.46 s (2H) [H2-3]; 3.64 t (2H) [H2-14]; 6.83 s (1H) [H-9]; 7.24 s (1H) [H-6]; 10.46 s (1H) [NH].

### Example 2a

### Preparation of 5-(2-Bromo-Acetyl)-6-Chloro-1,3-Dihydro-2H-Indol-5-On (IV)

40 g (0.3 mol) anhydrous AlCl₃ was suspended in 80 ml dichloro methane, it was cooled to a temperature between 0-10° C. and after 30 min stirring 9.6 ml (0.1 Imol) bromoacethyl-bromide was added dropwise, then 16.7 g 6-chloro-1,3-dihydro-2H-indol-5-on of Formula V was added, and the reaction mixture was stirred at a room temperature for 24 hours. The accomplishment of the reaction was checked by thin-layer chromatography. The reaction mixture was poured onto ice, the precipitated material was filtered out, then it was washed three times with 40 ml water, then with 20 ml methanol, and it was dried.

25.9 g 5-(2-bromo-acetyl)-6-chloro-1,3-dihydro-2H-indol-5-on of Formula IV was obtained. The material was characterised by the following NMR data: 1H NMR: 3.54 s (2H) [H2-3]; 4.79 s (2H) [H2-14]; 6.91 s (1H) [H-9]; 7.71 s (1H) [H-6]; 10.81 s (1H) [NH]. Example 2b.: Preparation of 5-(2-bromoethyl)-6-chloro-1,3-dihydro-2H-indol-2-one (III) 25.0 g (0.087 mol) 5-(bromoacethyl)-6-chloro-1,3-dihydro-2H-indol-5-one of Formula IV was dissolved in a mixture of 50 ml methanesulfonic acid and 50 ml dichloro methane, heated to the temperature of the boiling point, and then 30.5 ml (0.191 mol) trimethyl silane was added into it dropwise. After 30 min. stirring the accomplishment of the reaction was checked by thin-layer chromatography, and then the mixture was cooled to a temperature between 0-10° C., and 60 ml of water was added dropwise. The precipitated material was filtered out, then it was washed three times with 40 ml water, then with 20 ml methanol, and it was dried. 22.5 g 5-(2-bromoethyl)-6-chloro-1,3-dihydro-2H-indol-2-one of Formula III was obtained.

### Example 3

Preparation of 5-{2-[4-(1,2-Benzisothiazol-3-yl)-1-Piperazinyl]-Ethyl}-6-Chloro-1,3-Dihydro-2H-Indol-2-One (II)

38.4 g (0.175 mol) 3-piperazinyl-1,2-benzisothiazolt and 21.96 g (0.08 mol) 5-(2-bromoethyl)-6-chloro-1,3-dihydro-2H-indol-2-one are dissolved in 240 ml acetonitrile and boiled for 4 hours. The accomplishment of the reaction was controlled with a high-performance liquid chromatographic method. After cooling, the precipitated material was filtered out, washed with 50 ml acetonitrile. The moisty material was stirred in 240 ml distilled water at a temperature between 85-90° C. for an 1 hour, the solid was filtered out, then it was washed twice with 80 ml warm water, and it was dried.

28.1 g 5-{2-[4-(1,2-benzisothiazol-3-yl)-1 -piperazinyl]-ethyl}-6-chloro-1,3-dihydro-2H-indol-2-one was obtained.

This material was dissolved in 760 ml boiling tetrahydrofurane containing 7.5% water, 2.8 g charcoal and 2.8 g silica gel were added, and the mixture was boiled further for 30 min. After filtering the filtrate was evaporated at a reduced pressure to 80 ml volume, the concentrated mixture was stirred for 30 min in icy water. The filtered out material was washed with 20 ml cool tetrahydrofurane, and then it was dried.

23.4 g 5-{2-[4-(1,2-benzisothiazol-3-yl)-1-piperazinyl]-ethyl}-6-chloro-1,3-dihydro-2H-indol-2-one of Formula II was obtained. HPLC purity: 99.6%

1. A process for the preparation of pure ziprasidone base of Formula II reacting 3-piperazinyl-1,2-berizisothiazol of Formula VI with 5-(2-bromoethyl)-6-chloro-1,3-dihydro-2H-indol-2-one of Formula III 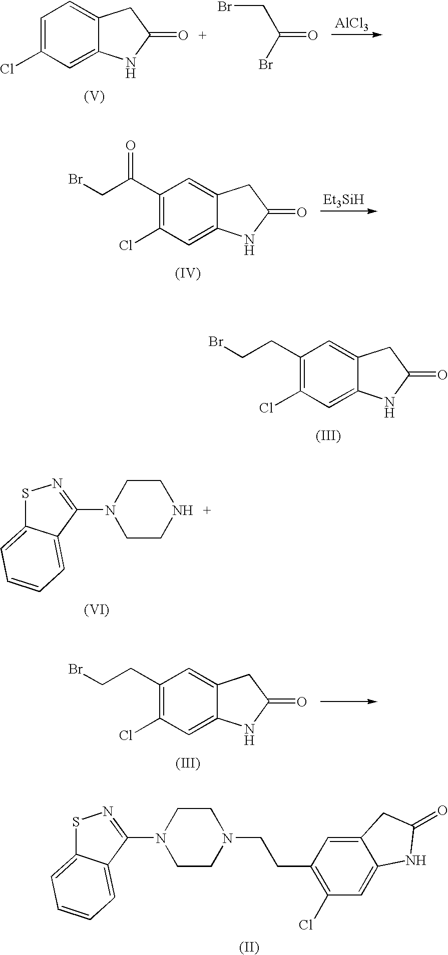
accomplishing the said reaction in an organic solvent, or in a mixture of organic solvents.

2. The process of claim 1 wherein the said reaction is accomplished in an aprotic solvent, preferably in acetonitrile and at the temperature of boiling.

3. The process of claim 1 wherein, the intermediate 5-(2-bromoethyl)-6-chloro-1,3-dihydro-2H-indol-2-one of Formula III is prepared from 5-(2-bromo-acetyl)-6-chloro-1,3-dihydro-2H-indol-5-on of Formula IV.

4. 5-(2-bromoethyl)-6-chloro-1,3-dihydro-2H-indol-2-one of Formula III.

5. A process for the preparation of 5-(2-bromoethyl)-6-chloro-1,3-dihydro-2H-indol-2one of Formula III, as defined in claim 1, using 5-(2-bromoacethyl)-6-chloro-1,3-dihydro-2H-indol-2-one of Formula IV as starting material.

6. The process of claim 2 wherein, the intermediate 5-(2-bromoethyl)-6-chloro-1,3-dihydro-2H-indol-2-one of Formula III is prepared from 5-(2-bromo-acetyl)-6-chloro-1,3-dihydro-2H-indol-5-on of Formula IV.
